(19) United States
(12) Patent Application Publication
(10) Pub. No.: US 2007/0078416 A1
(43) Pub. Date: Apr. 5, 2007

(54) Two-piece inline vascular access portal

(76) Inventor: Kenneth Eliasen, Wrentham, MA (US)

Correspondence Address: GROSSMAN, TUCKER, PERREAULT & PFLEGER, PLLC, 55 SOUTH COMMERICAL STREET, MANCHESTER, NH, 03101, US

(21) Appl. No.: 11/243,497

(22) Filed: Oct. 4, 2005

(51) Int. Cl. A61M 31/00

(52) U.S. Cl. 604/288.02

(57) ABSTRACT

An implantable inline vascular access portal having multiple fluid systems. Each fluid system of the access portal is isolated from the others. Each fluid system includes a fluid reservoir in fluid communication with a lumen of a multi-lumen stem. Fluid communication between one reservoir and the corresponding lumen of the stem is provided by an open channel defined in a body of the access portal. A septum is sealingly positioned over the fluid reservoirs and the open channel to seal the fluid reservoirs and the open channel in an isolated condition.

### FIELD

The present disclosure relates generally to a subcutaneously implantable vascular access port. More specifically, the present discloser pertains to implantable access ports having multiple fluid reservoirs isolated from one another.

### BACKGROUND

Direct access to the vascular system is a quick and effective way to administer a variety of drug therapies, provide nutrition, and/or sample blood. Currently, regular access to the vascular system is gained by using a device specifically designed for this task. Several types or families of these devices exist in the market today. Among them are needles, catheters and a group of devices known as implanted access portals.

Vascular access has evolved through the years to improve treatment of a number of chronic and non-chronic diseases. Needles have been used for many years to inject vaccines and antibiotics or withdraw blood. Although still widely used today, needles have several limitations that do not allow them to be used with all therapies. In the early 1970's the use of vascular access catheters was perfected and long term antibiotic, chemo, and nutritional therapies could be administered without having to change the access device and into a large enough vessel to allow the hemo-dilution required for some of the more toxic therapeutic drugs. This type of catheter provides a significant improvement over needles for long-term access, however their external segment is prone to infection and requires constant maintenance. The latest development in vascular access is the implanted access portal. These portals eliminate the need for an external segment and therefore do not have the drawbacks of catheters.

Although considered new technology in the vascular access arena, implanted access portals have existed in the market for over twenty years. Use of these products has increased dramatically during this period because they are generally the device of choice for long-term vascular access. They are particularly suited for long-term use because the entire device is implanted under the skin. Implantability is the key to the success of implantable access portals because implantation allows the patient to perform ordinary daily task such as bathing and swimming without worrying about harming an external segment of an access device or increasing the chance of infection. Thus the quality of life for the patient is improved and the clinician is presented with fewer device related complications.

Typically implanted access portals consist of a housing, a self-sealing septum, and an attachable or pre-connected catheter. Portal housings can be made of a variety of materials including plastic, metal, or a combination of both. The self-sealing septum is generally made of an elastomer such as silicone. Catheters are also generally made of a highly flexible material such as silicone or polyurethane. Different materials are used to manufacture the components to achieve certain desired characteristics in the portal. For example, plastic may be used because it is not radiopaque, and, therefore, the port will not show up on fluoroscopy.

Implanted access portals are also designed in such a way that their size (height and footprint), shape, and number of lumens are appropriate for the intended use. Number of lumens can be critical if a patient requires simultaneous infusion of incompatible solutions or isolation of blood sampling. As concurrent therapies become more popular the need for a wider variety of dual-lumen ports has increased.

### SUMMARY

The present disclosure is directed at an implantable inline vascular access portal which may include more than one fluid system. According to an embodiment, the more than one fluid systems may be isolated from each other. Each fluid system may generally include a fluid reservoir and a fluid pathway providing access to the fluid reservoir from outside the access portal. Access from outside the access portal may advantageously be achieved through a multi-lumen stem. The multiple lumens of the stem may each be in fluid communication with one of the fluid pathways.

According to one embodiment consistent with the present disclosure, an implantable inline vascular access portal may include a base having a plurality of fluid reservoirs. The portal may also include a stem having at least one lumen corresponding to each of the plurality of fluid reservoirs and in fluid communication therewith. An open channel in said base may provide fluid communication between at least one of the fluid reservoirs and a corresponding lumen of the stem. The access portal may further include at least one penetrable septum permitting ingress and egress of needles to the plurality of fluid reservoirs. The septum may be sealingly disposed over the plurality of fluid reservoirs and over the open channel in such a manner as to individually seal the plurality of fluid reservoirs.

According to another embodiment consistent with the present disclosure, an implantable inline vascular access portal may include a base including at least two fluid reservoirs, and a stem extending from the base. The stem may include at least two lumens, and each of the at least two fluid reservoirs may be in fluid communication with a respective one of the at least two lumens of the stem. Fluid communication for at least one of the fluid reservoirs may include an open channel. The access portal may further include a penetrable septum disposed over the at least two open fluid reservoirs and the open channel. A top member may be disposed over at least a portion of the septum and the base. The top member may include at least two access ways, which may provide penetrating access through the septum to the at least two fluid reservoirs.

### BRIEF DESCRIPTION OF THE DRAWINGS

Features and advantages of the claimed subject matter will be apparent from the following description particular embodiments consistent therewith, which description should be considered in conjunction with the accompanying drawings wherein:

FIG. 1 shows an assembly of an exemplary inline access port consistent with the present disclosure in an exploded perspective view;

FIG. 2 is a plan view of the base portion of the inline access port illustrated in FIG. 1; and

FIG. 3 is a perspective view the base portion of the exemplary inline access port illustrated in FIGS. 1 and 2.

### DESCRIPTION

FIG. 1 illustrates, in exploded perspective view, an assembly of an exemplary in-line vascular access port 100 consistent with the present disclosure including a top 102 a compound septum 104 and a base 106. As shown, the base 106 may include a stem 108 extending from one end of the base 106. The top 102 may desirably include a cut-out 110 generally corresponding to the position and size of the stem 108. The cut-out 110 may accommodate the stem 108 such that the top 102 may be coupled to the base 106, sandwiching the septum 104 in between the top 102 and the base 106.

The top 102 may be produced as an injection molded component, which may be formed from a biocompatible plastic. As mentioned above, the top 102 may be formed including cut-out 110 configured to accommodate the stem 108. Additionally, the top 102 may include access ways 112, 114 defined through the top 102, which may provide access to fluid reservoirs of the access portal 100. In one embodiment, the top 102 may include a reinforcing divider 116 between the access ways 112, 114. The reinforcing divider 116 may be formed by a thicker cross section and/or by a geometry having a greater section modulus than the nominal wall of the top 102.

The septum 104 may be formed from a needle penetrable, self sealing material that may provide ingress and egress of needles for delivering or retrieving fluid from the access portal 100. The septum 104 may be produced from materials such as silicone or other known biocompatible elastomer. As in the illustrated in-line access port 100, the septum 104 may be provided as a compound septum. That is, the septum 104 may include two, or more, individual septa 118 and 120. In addition to the individual septa 118, 120, the septum 104 may include a margin 122 extending around at least a portion of the perimeter of the septum 104. The septum 104 may be formed as a single block of elastomeric material. A compound septum 104, such as illustrated in FIG. 1, may allow a single component to individually seal more than one reservoir. This may facilitate assembly of the access port, reduce the overall part count, reduce the manufacturing processes, etc.

Advantageously, the septum 104 may include tactile and/or visual location markers (not shown). Such tactile or visual location markers may be configured to indicate the location of the septum 104 to permit efficient and accurate access to the septum 104 by a needle. Additionally, the tactile and/or visual location markers may be configured to distinguish the fluid reservoirs 202 and 204 from each other. Exemplary tactile or visual location markers may include dimples and/or protrusions on an upper surface of the septum 104.

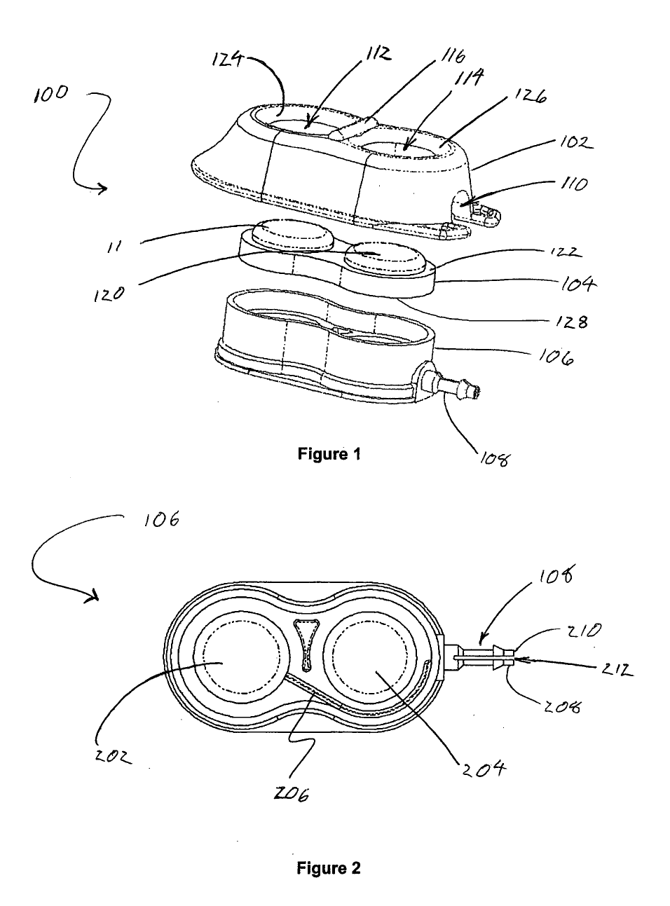

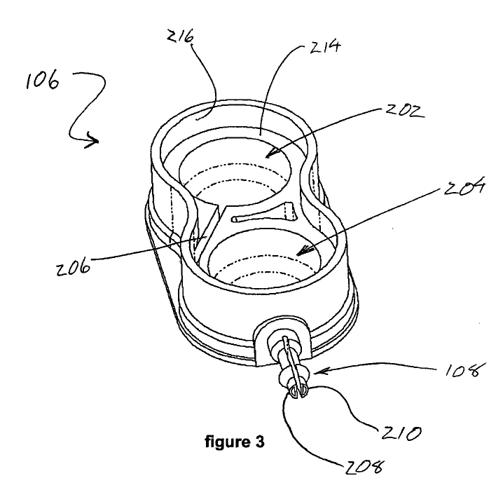

With further reference to FIGS. 2 and 3, the base 106 of the exemplary vascular access port 100 may include two fluid reservoirs 202 and 204. The base 106 may be formed from a biocompatible plastic, a metal, or an assembly of plastic and metal sub-components. Advantageously, the base 106 may be formed via injection molding, or other suitable forming technique.

A vascular access port according to the present disclosure may include a plurality of fluid systems, each of which may include a fluid reservoir and associated fluid pathways or lumens for conveying fluid to and/or from the fluid reservoir. The individual fluid systems, i.e., lumen and/or pathway and reservoir combination, may be isolated from each other. Isolating the individual fluid systems may, in some embodiments, prevent commingling of fluids in the respective systems. Each of the fluid reservoirs 202, 204 of the exemplary embodiment is shown to be in separate fluid communication with the stem 108. That is, each of the fluid reservoirs 202, 204 may be capable of communicating fluid with the stem 108 without being in fluid communication with the other fluid reservoir.

A first aspect of separate fluid communication from the reservoirs to the stem may include separate fluid channels from each respective fluid reservoir to the stem. For example, in the illustrated exemplary base 106, the distal reservoir 202 has a fluid path 206 extending from the reservoir 202 toward the stem 108. The fluid path 206 is directed around the proximal reservoir 204. The individual fluid paths may be molded in features of the base 106, which may provide direct communication with a corresponding reservoir. As most clearly shown in FIG. 3, providing fluid paths in direct communication with a corresponding reservoir may conveniently be provided in an economic manner using a conventional forming operation, such as injection molding. As shown, the separate fluid path 206 for the distal reservoir 202 is formed as an open channel. This arrangement may allow the use of a simple mold, e.g., which may not require side actions and the like to produce the path. However, use of other forming techniques is also contemplated herein.

According to another aspect, separate fluid communication with each of the fluid reservoirs may include the use of a bifurcated stem configuration. The exemplary stem 108 includes two separate lumens 208 and 210. Each of the separate lumens 208, 210 may provide isolated and/or separate fluid communication with a respective one of the reservoirs 202 and 204. The isolated fluid paths, extending from the respective fluid reservoirs, may be continued to any desired extent beyond and/or outside of the access portal. For example, in the illustrated embodiment, not only is the stem 108 bifurcated to provide two separate lumens, but the stem 108 additionally includes an axial slot 212 extending between the lumens 208 and 210. A bifurcated catheter, for example including a bisecting internal divider, may be used in combination with the stem 108 such that the internal divider of the catheter may be received in the slot 212. Accordingly, one lumen 208, 210 may be disposed in each respective half of the catheter.

As previously mentioned, the fluid systems of the access port 100 may be isolated, at least in part, by sealingly disposing the septum 104 over the reservoirs 202, 204 and/or the fluid pathway 206. According to an embodiment consistent with this aspect, the access portal 100 may be assembled such that the top 102 is coupled to the base 106 so as to sandwich the septum 104 therebetween. Each access way 112, 114 of the top 102 may be defined by a lip 124, 126 that is dimensioned to engage the margin 122 on a top surface of the septum 104. Similarly, the base 106 may include a rim 214 at least partially surrounding the reservoirs 202, 204. The rim 214 may be dimensioned corresponding to a bottom surface 128 of the septum 104. Each of the rim 214 of the base 106 and the bottom surface 128 of the septum 104 may have a generally planar configuration, which may facilitate sealing of the septum 104 to the base 106. In addition, the planar configuration of the bottom surface 128 of the septum 104 may simplify manufacture of the septum, and may also promote facile assembly of the access portal.

In an embodiment in which the septum 104 is formed from an elastomeric material, a compressive force applied to the septum 104 between the access way lips 124, 126 and the margin 214 of the base 106 may cause resilient deformation of the septum 104, which may facilitate achieving a fluid tight seal around the reservoirs 202, 204 and fluid pathways 206. The reinforcing divider 116 of the top 102 may assist in isolating the reservoirs 202, 204 from one another providing a line of compressive stress to the septum 104 extending between the reservoirs 202, 204. Achieving a fluid tight seal may be further aided by providing a bead, or similar feature on the rim of, and generally circumscribing, each reservoir. Such a bead or similar feature may provide a perimeter of higher localized sealing stress and/or deformation, which may improved the sealing characteristics.

As illustrated, the base 106 may include a side wall 216 extending upwardly from a periphery of the rim 214. The side wall 216 may be shaped and dimensioned to at least partially receive the septum 104. When the septum 104 is sandwiched between the top 102 and the base 106, the septum 104 may compress and resiliently deform. Resilient deformation of the septum 104 may include outward and/or laterally peripheral expansion of the septum 104. In one embodiment, the side wall 216 may restrict such lateral deformation of the septum 104. Accordingly, the septum 104 may experience greater compressive stress at the contact between the planar bottom surface 128 and the rim 214 and between the margin 122 and lips 124, 126. The increased compressive stress experienced at the contact between the septum and the rim 214 may enhance the sealing characteristics of the septum 104.

The top 102 may be coupled to the base 106 using a variety of methods, both permanent and releasable. Advantageously, the top 102 and the base 106 may be formed having interacting mechanical features, such as snap-fits, mating detents and undercuts, tongue and groove features, etc. Such mechanical features may allow simple snap-assembly of the access portal 100. Alternatively and/or additionally bonding and welding may also be employed for assembly of the access portal 100. Bonding and welding techniques may include adhesive bonding, sonic welding, thermal welding, solvent bonding, heat staking, and numerous other techniques that will be apparent to those having skill in the art.

The embodiments described herein have been set forth for the purposed of illustrating the various features and advantages of the present invention. The described exemplary embodiments are susceptible to modification and variation without departing from the spirit of the invention. For example, while the exemplary embodiment is directed at an in-line vascular access port having two separated reservoirs and associated fluid pathways, the number and configuration of reservoirs may readily be varied and adapted to suit different application, implant sites, etc. Additionally, numerous other techniques for sealing the individual fluid systems of the vascular access port will also be apparent to those having skill in the art. Therefore, the invention should not be considered to be limited by the description of exemplary embodiments above, but rather only by the appended claims.

What is claimed is:

1. An implantable inline vascular access portal comprising:
a base comprising a plurality of fluid reservoirs, a stem comprising at least one lumen corresponding to each of said plurality of fluid reservoirs and in fluid communication therewith, and an open channel defined in said base providing fluid communication between at least one of said plurality of fluid reservoirs and a corresponding lumen of said stem;
at least one needle penetrable septum, said septum comprising a planar bottom surface and sealingly disposed over said plurality of fluid reservoirs and said open channel to individually seal said plurality of fluid reservoirs.

2. The implantable vascular access portal according to claim 1, further comprising a top member disposed over at least a portion of said base and said septum, said top member comprising at least one access way corresponding to each of said plurality of fluid reservoirs.

3. The implantable vascular access portal according to claim 1, wherein said septum comprises a plurality of septa, wherein one of said septa is sealingly disposed over a respective one of said plurality of fluid reservoirs, and wherein at least one of said septa is sealingly disposed over said open channel.

4. The implantable vascular access portal according to claim 1, wherein said septum comprises at least one tactile or visual location marker differentiating at least one of said plurality of fluid reservoirs.

5. The implantable vascular access portal according to claim 1, comprising two fluid reservoirs disposed in an in-line configuration.

6. The implantable vascular access portal according to claim 1, wherein said stem comprises a bifurcated stem having two lumens.

7. An implantable inline vascular access portal comprising:
a base comprising at least two open fluid reservoirs, a stem extending from said base, said stem comprising at least two lumens, each of said at least two fluid reservoirs being in fluid communication with a respective one of said at least two lumens, and an open channel defined is said base and providing fluid communication between at least one of said fluid reservoirs and at least one of said lumens;
a penetrable septum comprising a planar bottom surface and disposed over said at least two open fluid reservoirs and said open channel; and
a top member disposed over at least a portion of said septum and said base, said top member comprising at least two access ways providing penetrating access through said septum to said at least two fluid reservoirs.

8. The implantable vascular access portal according to claim 7, wherein said base comprises a rim around said at least two open fluid reservoirs and said open channel, and wherein said septum is disposed in sealing contact with said rim.

9. The implantable vascular access portal according to claim 7, wherein said stem comprises a bifurcated stem comprising two lumens separated by a slot therebetween.
